(19) United States
(12) Patent Application Publication
(10) Pub. No.: US 2024/0209395 A1
(43) Pub. Date: Jun. 27, 2024

(54) MYELOID-SPECIFIC PROMOTER AND USE THEREOF

(71) Applicant: BEIJING MEIKANG GENO-IMMUNE BIOTECHNOLOGY CO., LTD., Beijing (CN)

(72) Inventors: Lung-Ji Chang, Beijing (CN); Haokun Yuan, Beijing (CN); Rui Zhang, Beijing (CN)

(21) Appl. No.: 18/557,159

(22) PCT Filed: Apr. 8, 2022
(86) PCT No.: PCT/CN2022/085852

§ 371 Date: Oct. 25, 2023

(30) Foreign Application Priority Data
Apr. 26, 2021 (CN) 202110455720.7
Jul. 6, 2021 (CN) 202110761757.2

(51) Int. Cl. C12N 15/86; A61K 48/00; C07K 14/47; C12N 5/0789; C12N 5/10

(52) U.S. Cl. CPC C12N 15/86; A61K 48/0058; C07K 14/47; C12N 5/0647; C12N 5/10; C12N 2510/00; C12N 2740/15021; C12N 2740/15043

(57) ABSTRACT

Provided are a myeloid-specific promoter and a use thereof. The myeloid-specific promoter includes a nucleic acid sequence as shown in SEQ ID NO: 1 or SEQ ID NO: 2. The myeloid-specific promoter shows specificity to myeloid tissues. It initiates a gene expression with high efficiency in myeloid cells, but with relative low efficiency in non-myeloid cells. As such, the myeloid-specific promoter regulates the specific expression of a gene in myeloid tissues. The myeloid-specific promoter and the CYBB gene are inserted into a lentiviral vector, and the constructed lentiviral expression vector shows specificity to myeloid tissues and can effectively restore the expression of gp91-phox protein and restore the generation function of ROS, which is of great significance for CGD treatment.

### TECHNICAL FIELD

The present disclosure belongs to the technical field of genetic engineering and relates to a myeloid-specific promoter and a use thereof.

### BACKGROUND

Chronic granulomatous disease (CGD) is a hereditary primary immunodeficiency disease affecting neutrophils and monocytes due to defects in functions of a nicotinamide adenine dinucleotide phosphate (NADPH) oxidase. The CGD is characterized by recurrent severe infections, inflammations and autoimmunity.

The NADPH oxidase consists of a membrane-bound protein and a cytoplasmic protein, which act synergistically, when phagocytes are activated, to help produce reactive oxygen species (ROS) to kill bacteria and fungi. A mutation in any of the five subunits of the NADPH oxidase will result in CGD syndrome. Approximately 67% of CGD cases are caused by defects of cytochrome b-245 beta chain (CYBB) gene on the X chromosome, which encodes transmembrane glycoprotein gp91-phox subunit.

At present, hematopoietic stem cell transplantation (HSCT) is the main method for treating CGD. HSCT needs thorough myeloablative preconditioning and needs to find an allogeneic human leukocyte antigen (HLA)-matched donor. However, in most cases, it is difficult for a patient to find a HLA-matched donor. Moreover, HSCT also has a risk of graft-versus-host disease (GVHD). In addition to the problems such as transplantation failure, high mortality and low donor chimerism, HSCT may also lead to immune rejection in the patient, which makes re-transplantation of hematopoietic stem cells very difficult.

Gene therapy refers to that a normal exogenous gene is introduced into target cells to correct or compensate for a defective gene and an abnormal gene for the purpose of treating a disease caused by the defective gene and the abnormal gene. Gene therapy for CGD began in the late 1990s when researchers attempted to use an adenovirus vector for CGD gene therapy. In addition to the inability to express the exogenous gene efficiently and continuously, the method also has the problem that the vector causes an immune response.

γ-retroviral vectors (γ-RVs) are also used for CGD treatment, but only a limited therapeutic effect is achieved. Kang et al. performed a gene therapy clinical trial on three X-CGD patients at the age of 19 to 23 by using a γ-RV to mediate gp91-Phox expression. After the cells were transduced with the viral vector, the initial percentage of positive cells was between 25% to 73%. In the seventh month after gene therapy, the percentage of functionally corrected cells in the peripheral blood of Patient 1 decreased from 24% to 1%. In the eleventh month after gene therapy, the percentage of functionally corrected cells in the peripheral blood of Patient 2 decreased from 4% to 0.03%. Four weeks later, corrected cells could not be detected in the peripheral blood of Patient 3 (see Hyoung, Jin, Kang, et al. Retroviral Gene Therapy for X-linked Chronic Granulomatous Disease: Results From Phase I/II Trial[J]. Molecular Therapy, 2011, 19, 2092-2101.).

Ravin et al. used CRISPR-Cas9 to repair a mutation in the CYBB gene in CGD patients. However, gene editing using the CRISPR-Cas9 system has the problems such as low efficiency and potential safety hazards. In addition, the method requires strict conditions, has a high cost and achieves an unstable result (see De Ravin et al. CRISPR-Cas9 gene repair of hematopoietic stem cells from patients with X-linked chronic granulomatous disease. Science Translational Medicine, 2017, 9, eaah 3480.).

In addition, the function of the NADPH oxidase is to produce ROS, and an overexpression of the ROS in cells may affect the normal functions of cells. Gene therapy can restore the production of the ROS in HCSs. The ROS has a great effect on a balance among processes such as resting, replication, proliferation and differentiation of the HSCs. A heterotopic expression of the NADPH oxidase mediated by a non-tissue-specific promoter will lead to the overexpression of the ROS in the HSCs. Excessive ROS could promote the apoptosis of resting HSCs, induce the HSCs to differentiate and weaken the self-renewal ability of the HSCs, resulting in apparent exhaustion of a HSC pool.

In summary, the adenovirus vector, the gamma-retroviral vector and the CRISPR-Cas9 system have defects in terms of safety, gene transfer efficiency and a long-term expression and the problems such as HSC apoptosis caused by the non-specific overexpression of the NADPH oxidase. Therefore, it is necessary to provide a viral vector having high gene transfer efficiency and suitable for stem cell modification to improve the treatment effect on CGD, which is of great significance in the field of CGD treatment.

### SUMMARY

The present disclosure provides a myeloid-specific promoter and a use thereof. The myeloid-specific promoter shows specificity to myeloid tissues. It initiates a gene expression with high efficiency in myeloid cells, but with relative low efficiency in non-myeloid cells.

In a first aspect, the present disclosure provides a myeloid-specific promoter which includes a nucleic acid sequence as shown in SEQ ID NO: 1 or SEQ ID NO: 2.

The myeloid-specific promoter of the present disclosure shows specificity to myeloid tissues. It initiates a gene expression with high efficiency in myeloid cells, but with relative low efficiency in non-myeloid cells. As such, the myeloid-specific promoter regulates the specific expression of a gene in myeloid tissues, which is of great significance in the field of gene therapy.

### SEQ ID NO: 1:

acttgtacagcttcacagggctccatgcttagaaggaccccacacttagtttaatgttctgctgtcatcatcttgatattcttaatttttaaata
aagggcctatcgttttcattttttactgggccttgcaaattatgtagctggttctgtatgccaggagagaagttggaagtaaaatggtattccag
gaccaggaggcattctggcagagtgaaagaacatgtgatttggagtccatggggatgggtttaaatttcagctttccactaatttgctttgtgat
actgagtatttccttttatccctcagaggctctgtttctcaattttgactacgggttttttcattagataatgtctcagttctggtattccaggt
ttccctcaattattctgggaaaacctccttgacccacaggcagagcctagggcagccaggtgctttctactctctctctctctgcagcttggaaa
gttagtgtctgttgaaggtcagctgggagttggtggaggcagggcagtggcctgctactattgctgcagtagcagaccctttcacaacagcattg
ttttgtcattttgcatccagatttccgttggctaacctcagtcttatcttcctcatttctgtttcctgttgaagacaccaagggcccttcaaaac
acagaagcttcttgctcacggcagaaagcccaattccatctggcccctgcaggttggctcagcactggggaatcagagtcccctccatgaccaag
gcaccactccactgacag.

### SEQ ID NO: 2:

tagccatttgctggaccaaatctggagggagaacccctaaaacccctaagtgaggttgcccagggggttgtccccaggtggggggaag
caggggagagaaaatggtagccatttttacattgttttgtatagtatttattgattcaggaaacaaacacaaaattctgattataatgacttgga
aactgcctgtttgggcttctcatttcttacctccccttccctctcccacctgctactgggtgcatctctgctccccccttccccagcagatggtt
acctttgggctgttgctttcttgtcaccatctgagttctcagacgctggaaagccatgttctcggctctgtgaatgacaatgctgactggagtgc
tgcccctctgtaaagggctgggtgtggatggtcacaagcccctcacatgcctcagccaagaggaagtagtacaggggtcagcccagaggtccagg
ggaaaggagtggaaaccgatttccccaccaagggaggggcctgtacctcagctgttcccatagctacttgccacaactgccaagcaagtttcgct
gagtttgacacatggaaccctgtggatcaactgccctaggactccgtttgcacccatgtgacactgttgactttgccctgatgaagcagggccaa
cagtcccctaacttaattacaaaaactaatgactaagagagaggtggctagagctgaggcccctgagtcaggctgtggggggatcatctccagta
caggaagtgagactttcatttcctcctttccaagagagggctgagggagcagggttgagcaactggtgcagacagcctagctggactttgggtga
ggcggttcagcc.

In a second aspect, the present disclosure provides a recombinant expression vector which includes the myeloid-specific promoter according to the first aspect.

In some specific embodiments, the recombinant expression vector includes a viral vector or a plasmid vector containing the myeloid-specific promoter according to the first aspect. Preferably, the viral vector includes a pTYF lentiviral vector.

Preferably, the recombinant expression vector further includes a CYBB gene.

Preferably, the CYBB gene includes a nucleic acid sequence as shown in SEQ ID NO: 3.

### SEQ ID NO: 3:

atggggaactgggctgtgaatgaggggctctccatttttgtcattctggtttggctggggttgaacgtcttcctctttgtctggtattaccgggt
ttatgatattccacctaagttcttttacacaagaaaacttcttgggtcagcactggcactggccagggcccctgcagcctgcctgaatttcaact
gcatgctgattctcttgccagtctgtcgaaatctgctgtccttcctcaggggttccagtgcgtgctgctcaacaagagttcgaagacaactggac
aggaatctcacctttcataaaatggtggcatggatgattgcacttcactctgcgattcacaccattgcacatctatttaatgtggaatggtgtgt
gaatgcccgagtcaataattctgatccttattcagtagcactctctgaacttggagacaggcaaaatgaaagttatctcaattttgctcgaaaga
gaataaagaaccctgaaggaggcctgtacctggctgtgaccctgttggcaggcatcactggagttgtcatcacgctgtgcctcatattaattatc
acttcctccaccaaaaccatccggaggtcttactttgaagtcttttggtacacacatcatctctttgtgatcttcttcattggccttgccatcca
tggagctgaacgaattgtacgtgggcagaccgcagagagtttggctgtgcataatataacagtttgtgaacaaaaaatctcagaatggggaaaaa
taaaggaatgcccaatccctcagtttgctggaaaccctcctatgacttggaaatggatagtgggtcccatgtttctgtatctctgtgagaggttg
gtgcggttttggcgatctcaacagaaggtggtcatcaccaaggtggtcactcaccctttcaaaaccatcgagctacagatgaagaagaaggggtt
caaaatggaagtgggacaatacatttttgtcaagtgcccaaaggtgtccaagctggagtggcacccttttacactgacatccgcccctgaggaag
acttctttagtatccatatccgcatcgttggggactggacagaggggctgttcaatgcttgtggctgtgataagcaggagtttcaagatgcgtgg
aaactacctaagatagcggttgatgggccctttggcactgccagtgaagatgtgttcagctatgaggtggtgatgttagtgggagcagggattgg
ggtcacacccttcgcatccattctcaagtcagtctggtacaaatattgcaataacgccaccaatctgaagctcaaaaagatctacttctactggc
tgtgccgggacacacatgcctttgagtggtttgcagatctgctgcaactgctggagagccagatgcaggaaaggaacaatgccggcttcctcagc
tacaacatctacctcactggctgggatgagtctcaggccaatcactttgctgtgcaccatgatgaggagaaagatgtgatcacaggcctgaaaca
aaagactttgtatggacggcccaactgggataatgaattcaagacaattgcaagtcaacaccctaataccagaataggagttttcctctgtggac
ctgaagccttggctgaaaccctgagtaaacaaagcatctccaactctgagtctggccctcggggagtgcatttcattttcaacaaggaaaacttc
taa.

Preferably, the myeloid-specific promoter initiates the expression of the CYBB gene.

In some specific embodiments, a lentiviral vector is used for transduction of blood stem cells or somatic cells with high efficiency, high stability and high safety so that the gene can be transferred efficiently during gene therapy. Meanwhile, the myeloid-specific promoter is used so that the lentiviral vector specifically expresses the CYBB gene in myeloid cells, thereby effectively treating the chronic granulomatous disease caused by gene mutation on the X chromosome.

In a third aspect, the present disclosure provides a recombinant lentivirus containing the recombinant expression vector according to the second aspect.

In a fourth aspect, the present disclosure provides a recombinant cell containing the myeloid-specific promoter according to the first aspect.

In some specific embodiments, the recombinant cell contains the recombinant expression vector according to the second aspect.

In some specific embodiments, the recombinant cell contains the recombinant lentivirus according to the third aspect.

In a fifth aspect, the present disclosure provides a method for preparing the recombinant cell according to the fourth aspect, which includes:
introducing the recombinant expression vector according to the second aspect or the recombinant lentivirus according to the third aspect into a host cell to obtain the recombinant cell.

Preferably, the introduction is carried out by a method which includes any one of electrical gene transfer, a viral vector system, a non-viral vector system or gene gun injection.

Preferably, the host cell includes a hematopoietic stem cell.

Preferably, the method includes:
(1) constructing a lentiviral vector;
(2) co-transfecting the lentiviral vector in step (1) and a packaging plasmid or packaging plasmids into a mammalian cell for lentiviral vector packaging; and
(3) introducing the packaged lentiviral vector in step (2) into a host cell to obtain the recombinant cell.

Preferably, the step (1) of constructing a lentiviral vector includes: inserting the myeloid-specific promoter according to the first aspect and a CYBB gene into a pTYF lentiviral vector.

Preferably, the packaging plasmids in step (2) include pNHP and pHEF-VSVG.

Preferably, the mammalian cell in step (2) includes a 293T cell.

In a sixth aspect, the present disclosure provides a pharmaceutical composition which includes any one or a combination of at least two of the myeloid-specific promoter sequences according to the first aspect, the recombinant expression vector according to the second aspect, the recombinant lentivirus according to the third aspect or the recombinant cell according to the fourth aspect.

Preferably, the pharmaceutical composition further includes any one or a combination of at least two of a pharmaceutically acceptable carrier, excipient or diluent.

In a seventh aspect, the present disclosure provides a use of the myeloid-specific promoter according to the first aspect, the recombinant expression vector according to the second aspect, the recombinant lentivirus according to the third aspect, the recombinant cell according to the fourth aspect or the pharmaceutical composition according to the sixth aspect in the preparation of a drug for treating a disease.

Preferably, the disease includes CGD.

Compared with the existing art, the present disclosure has the following beneficial effects:

(1) The myeloid-specific promoter of the present disclosure shows specificity to myeloid tissues. It initiates a gene expression with high efficiency in myeloid cells, but with relative low efficiency in non-myeloid cells. As such, the myeloid-specific promoter regulates the specific expression of a gene in myeloid tissues.

(2) In the present disclosure, the myeloid-specific promoter is inserted into a lentiviral vector to obtain a lentiviral vector which has high transduction efficiency, high stability and high safety and can perform specific expression in myeloid cells.

(3) In the present disclosure, the myeloid-specific promoter and the CYBB gene are inserted into a lentiviral vector, and the constructed lentiviral expression vector shows specificity to myeloid tissues and can effectively restore the expression of gp91-phox protein and restore the generation function of ROS, which is of great significance for CGD treatment.

### BRIEF DESCRIPTION OF DRAWINGS

FIG. 1 is a diagram illustrating the viral vector copy number (VCN) in C57 mouse bone marrow HSCs.

FIG. 2 is a diagram illustrating the expression of GFPs in C57 mouse HSCs on Day 5 and Day 14 after transfected with lentiviruses.

FIG. 3 is a diagram illustrating expression percentages of GFPs in C57 mouse HSCs on Day 5 and Day 14 after transduced with lentiviruses.

FIG. 4 is a diagram illustrating results of the expression of the CYBB gene in X-CGD mouse HSCs.

FIG. 5 is a diagram illustrating generation levels of ROS in X-CGD mouse HSCs.

FIG. 6 is a diagram illustrating percentages of mouse HSCs that differentiated into myeloid cells on Day 14 of differentiation induction.

FIG. 7 is a diagram illustrating results of an Escherichia coli-phagocytizing experiment.

FIG. 8 is a diagram illustrating results of VCN in X-CGD mouse HSCs transduced with lentiviruses.

FIG. 9 is a diagram illustrating results of the expression of the CYBB gene in mouse cells in vivo.

FIG. 10 is a diagram illustrating results of the generation level of ROS in mouse cells in vivo.

### DETAILED DESCRIPTION

To further elaborate on the technical means adopted and effects achieved in the present disclosure, the present disclosure is further described below in conjunction with examples and drawings. It is to be understood that the specific examples set forth below are intended to explain the present disclosure and not to limit the present disclosure.

Experiments without specific techniques or conditions noted in the examples are conducted according to techniques or conditions described in the literature in the art or a product specification. The reagents or instruments used herein without manufacturers specified are conventional products commercially available from proper channels.

### Example 1

A recombinant lentivirus was prepared. The method for preparing the recombinant lentivirus includes steps described below.

### (1) Construction of a Lentiviral Vector

1) A pTYF lentiviral vector was modified by mutating wild-type 5′ splice donor site GT into CA, and deleting the enhancer in the U3 region. For a specific modification method, see “Cui Y, Iwakuma T, Chang L J. Contributions of Viral Splice Sites and cis-Regulatory Elements to Lentivirus Vector Function[J]. Journal of Virology, 1999, 73 (7): 6171.”

### Wild-Type 5′ Splice Donor Site SEQ ID NO: 4:

GGCAAGAGGCGAGGGGCGGCGACTGGTGAGTACGCCAAAAATTTTGACTAGCGGA GGCTA;
Mutant 5′ splice donor site SEQ ID NO: 5:
GGCAAGAGGCGAGGGGCGGCGACTGCAGAGTACGCCAAAAATTTTGACTAGCGGA GGCTA.

2) A cDNA sequence of CYBB gene (SEQ ID NO: 3), an miR223 promoter sequence (SEQ ID NO: 1) and a CD68 promoter sequence (SEQ ID NO: 2) were synthesized, and these sequences were correspondingly ligated into lentiviral vector TYF through restriction enzyme sites to obtain an miR223+CYBB lentiviral vector and a CD68+CYBB lentiviral vector.

### (2) Lentivirus Packaging and Concentration

1) 293T cells were inoculated in a fresh Dulbecco's modified eagle's medium (DMEM) containing 10% fetal bovine serum (FBS) and incubated for 17 h.

2) The two lentiviral vectors prepared in step (1), DMEM, pNHP and pHEF-VSV-G were added to a sterile centrifuge tube in sequence, vortexed and mixed, and then a Superfect transfection reagent (QIAGEN) was added to the centrifuge tube. The system was allowed to stand at room temperature for 8 min.

3) The mixture prepared in the centrifuge tube was added dropwise to 293T cells and incubated for 5 h at 37° C. under 5% CO₂.

4) The cell culture medium was discarded, and the cells were rinsed and added with a fresh medium to continue the culture.

5) The cell culture medium was collected, the cells were rinsed, and the culture medium was replaced with a fresh culture medium. The fresh medium was incubated in a 5% CO₂ incubator overnight. Then, the cell culture medium was collected and stored at −80° C.

6) The packaged lentivirus was centrifuged for 5 min at 1000×g, cell fragments were removed and the remaining lentivirus was stored at −80° C.

7) The supernatant of the lentivirus was added to a centrifuge filter tube and centrifuged at 2500×g for 30 min. The concentrated virus was collected into a centrifuge tube and stored at −80° C. to obtain lentiviruses LV-miR223 and LV-CD68 expressing CYBB.

### Example 2

Gene transfer efficiency and promoter specificity were verified in C57 mouse HSCs.

C57 mouse bone marrow HSCs were separately transduced with CYBB-expressing lentiviruses LV-EF1α, LV-miR223, LV-CD68 and LV-VEC, where LV-EF1α was a lentivirus carrying a widely expressed strong mammalian EF1α promoter, LV-VEC was a lentivirus carrying an endothelial cell-specific promoter, and cells transduced with no lentiviruses were used as a negative control (NC).

C57 mouse HSCs were transduced by the method described below.

(1) Bone marrow was taken from the tibia of a C57 mouse, and HSCs were isolated and extracted from the bone marrow using EasySep™ Mouse Hematopoietic Progenitor Cell Isolation Kit available from STEMCELL Technologies.

(2) 1×10⁶ mouse HSCs were resuspended in 100 μL medium (StemSpan SFEM Medium available from STEMCELL Technologies) containing cytokines (including 50 ng/mL stem cell growth factor (SCF), 50 ng/mL FMS-like tyrosine kinase 3 ligand (FLT3-L), 10 ng/mL interleukin 6 (IL6) and 50 ng/mL thrombopoietin (TPO) available from Biotech Company) and stimulated and incubated for 17 h.

(3) 50 μL medium was discarded, and 50 μL fresh medium containing cytokines was added to resuspend the cells. 8 μg/mL polybrene was added, and the lentivirus was added and mixed. The multiplicity of infection (MOI) of the transfection was 200. The cells were transfected once a day, twice in total. Centrifuged at 100×g at room temperature for 100 min.

(4) After the transduction was completed, the cells were collected and induced by 20 ng/ml murine granulocyte colony-stimulating factor (an mG-CSF cytokine available from PeproTech, Inc.) to differentiate into myeloid cells. On Day 5 and Day 14, cells were collected and measured for the expression of green fluorescent proteins (GFPs) through flow cytometry.

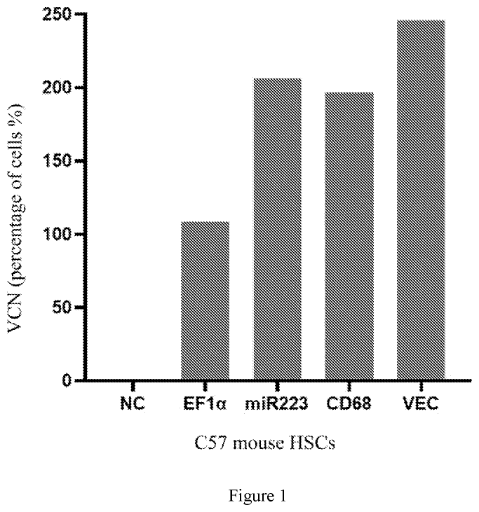

After the virus transduction, q-PCR was used to determine the VCN in the cells. The results are shown in FIG. 1. The viral VCNs of the lentiviruses LV-miR223 and LV-CD68 after the transduction were 206.33% and 196.87%, respectively, indicating that the lentiviral vector containing a myeloid-specific promoter constructed in the present disclosure can be effectively transfected into cells and meet the requirements of gene therapy.

The lentiviral vector carried a GFP fluorescent gene. Photos were taken and the expression of the lentiviral vector was analyzed by measuring the expression percentage of GFPs. The expression of GFPs on Day 5 (the cells were not differentiated into myeloid cells (undiffs)) and the expression of GFPs on Day 14 (the cells were differentiated into myeloid cells (diffs)) were compared, and the myeloid specificity of two promoters was analyzed.

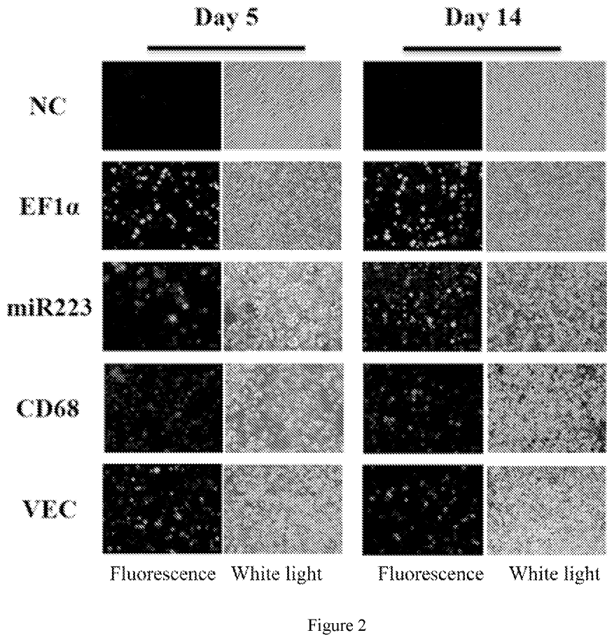

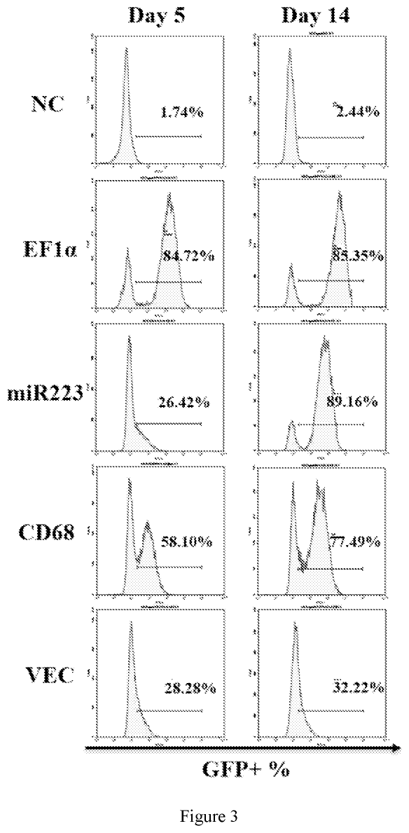

The results are shown in FIGS. 2 and 3. FIG. 2 is a diagram illustrating the expression of GFPs in cells on Day 5 and Day 14 after induced differentiation, where the first column is a fluorescent photograph, and the second column is a white light photograph. FIG. 3 is a diagram illustrating expression percentages of GFPs in C57 mouse HSCs on Day 5 and Day 14 after transduced with lentiviruses. The expression percentages of GFPs in the undiff cells and the diff cells in the EF1α group were 84.72% and 85.35%, respectively, which are similar. The expression percentages of GFPs in the undiff cells and the diff cells in the VEC group were 28.28% and 32.22%, respectively, which are similar. The expression percentages of GFPs mediated by miR223 in the undiff cells and the diff cells were 26.42% and 89.16%, respectively, which have a significant difference. The expression percentages of GFPs mediated by CD86 in the undiff cells and the diff cells were 58.01% and 77.49%, respectively, which have a significant difference. It can be seen that the miR223 promoter and the CD86 promoter initiate gene expression in the myeloid cells with higher efficiency than in non-myeloid cells, that is, the miR223 promoter and the CD86 promoter have myeloid specificity. Moreover, the miR223 promoter has a greater difference in expression, that is, the miR223 promoter has higher specificity.

### Example 3

Gene transfer efficiency was verified and the abilities of promoters to initiate the expression of CYBB gene and restore functions of NADPH oxidase and the specificity of the promoters were compared in HSCs of CGD mice (X-CGD mice, B6.129S-Cyb btm1Din/J).

X-CGD mouse HSCs were transduced by the method described below.

(1) Bone marrow was taken from the tibia of a X-CGD mouse, and HSCs were isolated and extracted from the bone marrow using EasySep™ Mouse Hematopoietic Progenitor Cell Isolation Kit available from STEMCELL Technologies.

(2) 1×10⁶ mouse HSCs were resuspended in a 100 μL medium (StemSpan SFEM Medium available from STEMCELL Technologies) containing cytokines (including 50 ng/mL SCF, 50 ng/mL FLT3-L, 10 ng/ml IL6 and 50 ng/mL TPO available from Biotech Company) and stimulated and incubated for 17 h.

(3) 50 μL medium was discarded, and 50 μL fresh medium containing cytokines was added to resuspend the cells. 8 μg/mL polybrene was added, and the viral vector was added and mixed. The MOI of the transduction was 200. The cells were transduced once a day, twice in total. Centrifuged at 100×g at room temperature for 100 min.

(4) After the transduction was completed, the cells were collected and induced by 20 ng/ml murine granulocyte colony-stimulating factor (an mG-CSF cytokine available from PeproTech, Inc.) to differentiate into myeloid cells.

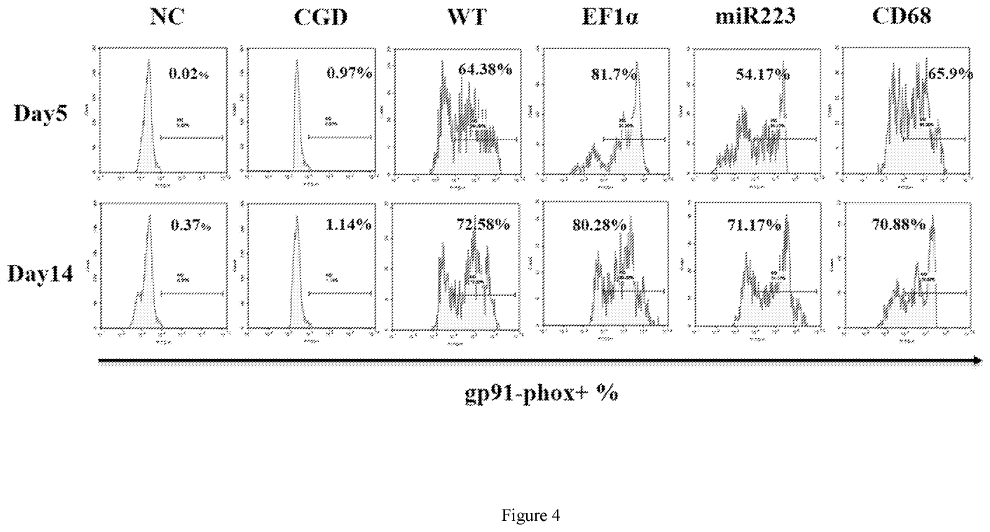

The expression of the CYBB gene (expressing gp91-phox protein) was detected on Day 5 and Day 14, respectively, that is, percentages of gp91-phox-positive cells on Day 5 (undiff) and Day 14 (diff) were measured through flow cytometry. The results are shown in FIG. 4, where NC represents X-CGD mouse HSCs transduced with no lentivirus, CGD represents X-CGD mouse HSCs transduced with no lentivirus but stained with an anti-gp91-phox antibody, and WT represents wild-type mouse cells.

As can be seen from FIG. 4, the expression percentages of gp91-phox protein in diff cells and undiff cells in the WT group were 72.58% and 64.38%, respectively; the expression percentages of gp91-phox protein in diff cells and undiff cells in the EF1α group were 80.28% and 81.7%, respectively; the expression percentages of gp91-phox protein in diff cells and undiff cells in the miR223 group were 71.17% and 54.17%, respectively; and the expression percentages of gp91-phox protein in diff cells and undiff cells in the CD68 group were 70.8% and 65.9%, respectively. In summary, the miR223 promoter and the CD86 promoter initiate gene expression in the myeloid cells with higher efficiency than in non-myeloid cells, that is, the miR223 promoter and the CD86 promoter have myeloid specificity. Moreover, the miR223 promoter has higher specificity.

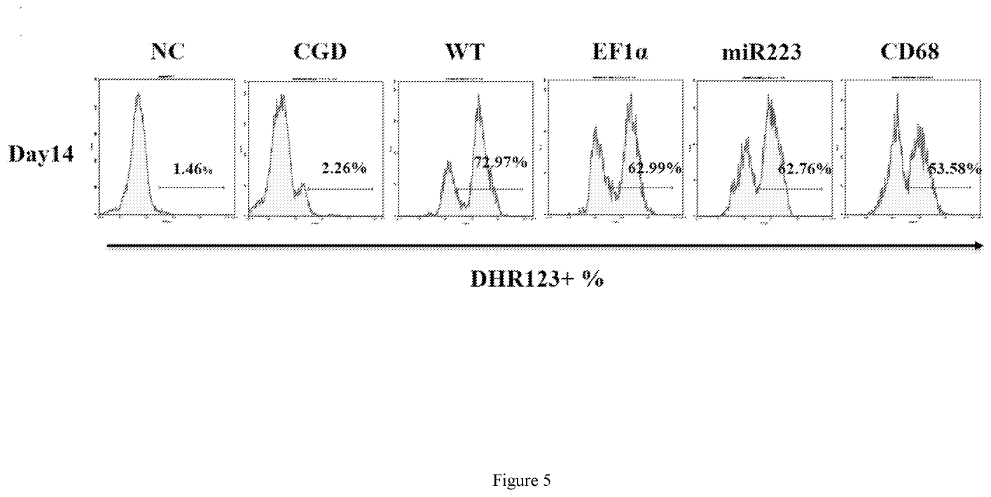

The cells were stimulated by phorbol ester (PMA) and stained with dihydrorhodamine (DHR123), and the generation level of ROS in the cells was measured through flow cytometry on Day 14 to further verify the expression of CYBB gene. The results are shown in FIG. 5. The DHR123+% in the WT group was 72.97%, the DHR123+% in the EF1α group was 62.99%, the DHR123+% in the MiR223 group was 62.76%, and the DHR123+% in the CD68 group was 53.58%. It can be seen that the lentiviral vector constructed in the present disclosure can effectively express the CYBB gene, that is, the lentiviral vector can effectively restore the generation level of ROS in CGD cells to a level close to that of ROS in normal wild-type cells.

### Example 4

The effect of the viral vector on the differentiation ability of X-CGD mouse HSCs was determined.

X-CGD mouse HSCs were transduced by the method described below.

(1) Bone marrow was taken from the tibia of a X-CGD mouse, and HSCs were isolated and extracted from the bone marrow using EasySep™ Mouse Hematopoietic Progenitor Cell Isolation Kit available from STEMCELL Technologies.

(2) 1×10⁶ mouse HSCs were resuspended in a 100 μL medium (StemSpan SFEM Medium available from STEMCELL Technologies) containing cytokines (including 50 ng/mL SCF, 50 ng/mL FLT3-L, 10 ng/ml IL6 and 50 ng/mL TPO available from Biotech Company) and stimulated and incubated for 17 h.

(3) 50 μL medium was discarded, and 50 μL fresh medium containing cytokines was added to resuspend the cells. 8 μg/mL polybrene was added, and the viral vector was added and mixed. The MOI of the transduction was 200. The cells were transduced once a day, twice in total. Centrifuged at 100×g at room temperature for 100 min.

(4) After the transfection was completed, the cells were collected, inoculated in a fresh RPMI1640 medium containing 20% FBS and induced to differentiate by 20 μg/mL murine granulocyte colony-stimulating factor (an mG-CSF cytokine available from PeproTech, Inc.). The medium was replaced every two days, and the cells were cultured for 14 days.

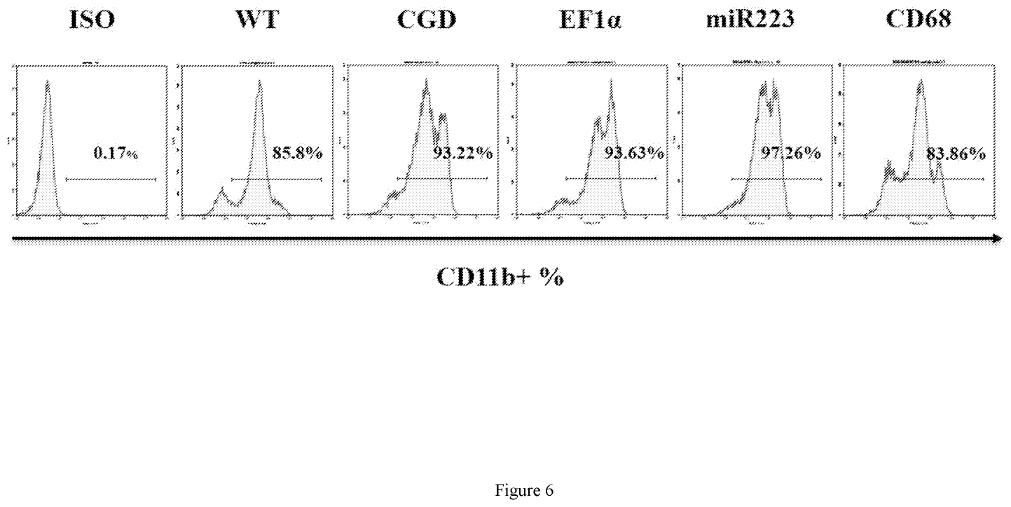

Mouse HSCs can be induced by the murine granulocyte colony-stimulating factor to differentiate into myeloid cells (phagocytes and neutrophils). Since CD11b is an important marker of the myeloid cells, the percentage of CD11b-positive cells was measured through flow cytometry in order to determine cell differentiation. The results are shown in FIG. 6, where cells transduced with no lentivirus and treated with an isotype antibody were used as a negative control (ISO).

As can be seen from FIG. 6, the CD11b+% in the WT group was 85.8%, the CD11b+% in the miR223 group was 97.26%, and the CD11b+% in the CD68 group was 83.86%, indicating that the lentiviral vector constructed in the present disclosure will not affect the differentiation ability of cells transduced with the lentiviral vector, that is, the lentiviral vector is safe.

### Example 5

The effect of the lentiviral vector on the phagocytic function of X-CGD mouse HSCs after differentiation was determined.

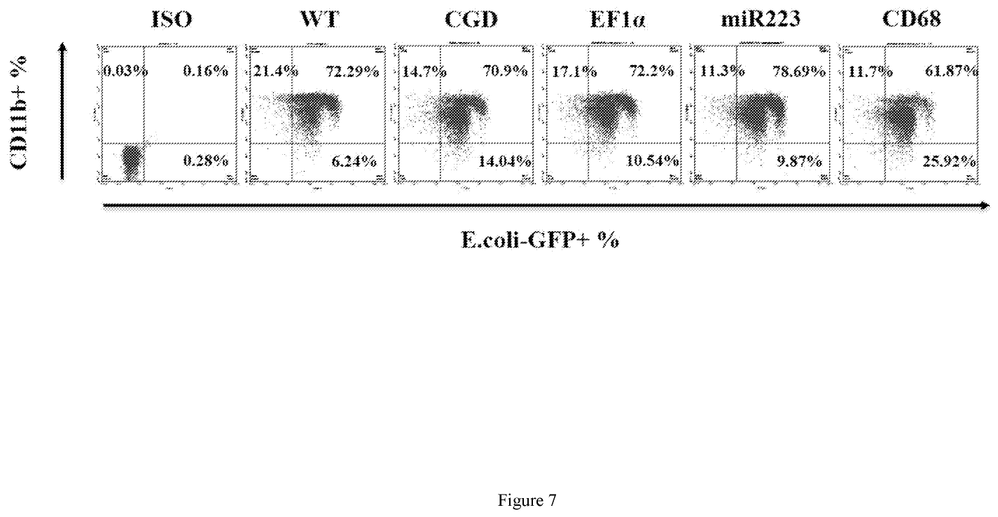

The lentiviral transduction and induced differentiation experiments were the same as that described in Example 4. Cells that had been completely induced to differentiate were taken, washed using PBS and counted, and an experiment was carried out according to 1:100 of cell/E. coli-GFP+. The medium was a fresh RPMI1640 medium containing 20% FBS, and the cells were cultured for 2.5 h in total and washed using PBS. The fluorescence of fluorescein isothiocyanate (FITC) was tested through flow cytometry. The results are shown in FIG. 7.

As can be seen from FIG. 7, in the wild-type cells (WT group), the CD11b+% was 83.27% and the E. coli-GFP+% was 87.07%; in the cells transduced with the lentivirus LV-miR223 (miR223 group), the CD11b+% was 89.76% and the E. coli-GFP+% was 84.59%; and in the cells transduced with the lentivirus LV-CD68 (CD68 group), the CD11b+% was 83.99% and the E. coli-GFP+% was 82.77%. It can be seen from the comparison that after the lentiviruses designed in the present disclosure are transfected into the HSCs, the lentiviruses have no effect on the differentiation of HSCs into myeloid cells and the phagocytic function of the differentiated cells. Therefore, the lentiviral vectors designed in the present disclosure are proved to be safe.

### Example 6

The ability of the lentiviral vector to correct the functions of phagocytes and neutrophils was evaluated in X-CGD mice.

1.5×10⁶ X-CGD mouse HSCs were taken and separately transduced with lentiviruses LV-miR223, LV-CD86 and LV-EF1α in vitro with an MOI of 200. The X-CGD mouse HSCs were transduced by the same method as those in Example 4.

Myeloablative preconditioning was conducted on X-CGD mice through irradiation at a radiation dose of 4.5 Gy. On Day 4 after the treatment, the above cells transduced with the lentiviruses were transplanted via tail veins. Four weeks later, the peripheral blood was taken for detection, including detecting the VCN through qPCR, detecting the expression of the CYBB gene through flow cytometry and measuring the generation level of ROS in the cells stained with DHR123.

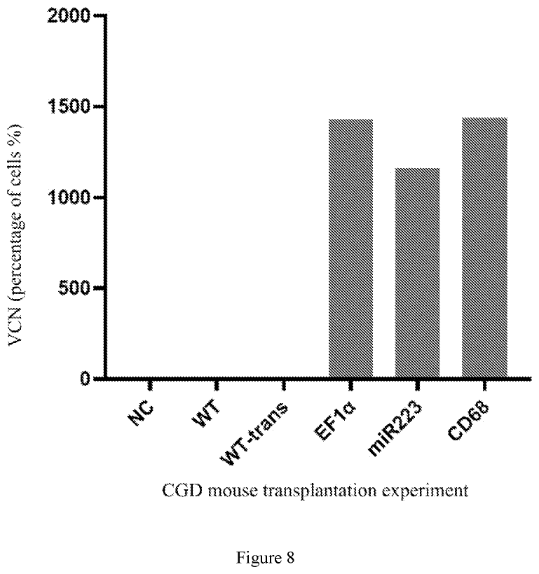

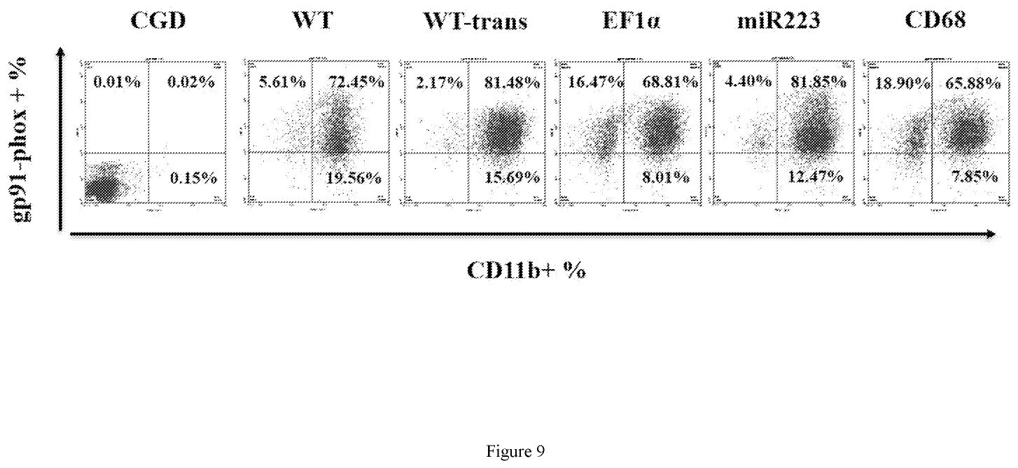

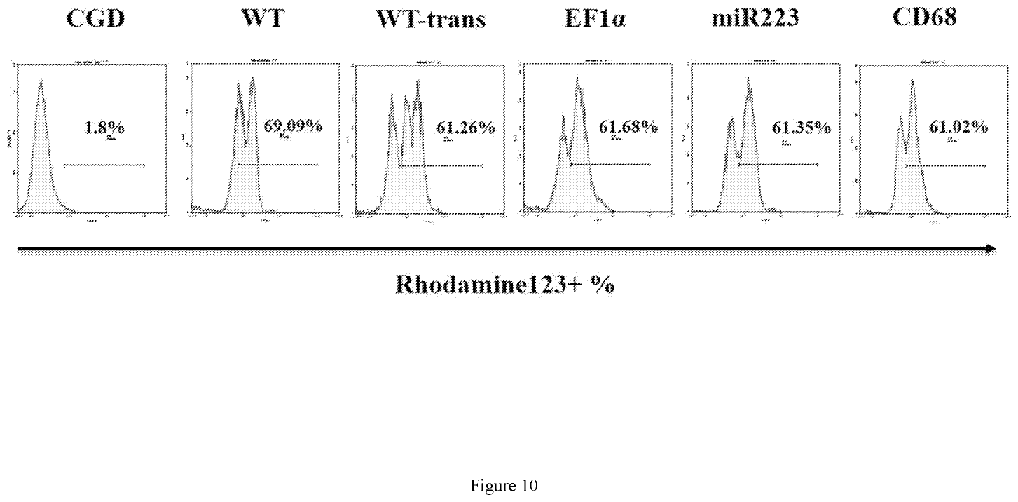

FIG. 8 is a diagram illustrating VCN results. FIG. 9 is a diagram illustrating results of the expression of the CYBB gene. FIG. 10 is a diagram illustrating results of the generation level of ROS in cells. FIG. 8 shows that lentiviruses can be efficiently transfected. It can be seen from FIGS. 9 and 10 that in the isotype wild-type C57 mice (WT group), the gp91-phox+% was 59.37% and the Rhodamine123+% was 68.59%; in the CGD mice transplanted with wild-type C57 mouse HSCs (WT-trans group), the gp91-phox+% was 57.14% and the Rhodamine123+% was 61.26%; in the CGD mice transplanted with HSCs transduced with LV-miR223 (miR223 group), the gp91-phox+% was 58.98% and the Rhodamine123+% was 58.29%; and in the CGD mice transplanted with HSCs transduced with LV-CD68 (CD68 group), the gp91-phox+% was 58.29% and the Rhodamine123+% was 61.35%. It can be seen from the comparison that after the HSCs transduced with the lentiviral vectors designed in the present disclosure are transplanted back into the X-CGD mice, the lentiviral vectors can effectively restore the expression of gp91-phox proteins and the generation function of ROS. Therefore, the lentiviral vectors designed in the present disclosure are proved to be effective.

In summary, in the present disclosure, the myeloid-specific promoter and the CYBB gene are inserted into the lentiviral expression vector. The constructed lentiviral expression vector has high transduction efficiency, stable expression ability, safety and myeloid specificity. The lentiviral expression vector is effectively expressed in the myeloid cells and can effectively restore the expression of gp91-phox proteins and restore the generation function of ROS, which is of great significance for CGD treatment.

The applicant has stated that although the detailed method of the present disclosure is described through the examples described above, the present disclosure is not limited to the detailed method described above, which means that implementation of the present disclosure does not necessarily depend on the detailed method described above. It should be apparent to those skilled in the art that any improvements made to the present disclosure, equivalent replacements of raw materials of the product of the present disclosure, additions of adjuvant ingredients to the product of the present disclosure, and selections of specific manners, etc., all fall within the protection scope and the disclosure scope of the present disclosure.

1. A myeloid-specific promoter, comprising at least 90% of a nucleic acid sequence as shown in SEQ ID NO: 1 or SEQ ID NO: 2.

2. A recombinant expression vector, comprising the myeloid-specific promoter according to claim 1.

3. The recombinant expression vector according to claim 2, wherein the recombinant expression vector further comprises a cytochrome b-245 beta chain (CYBB) gene;
and
the myeloid-specific promoter initiates the expression of the CYBB gene.

4. A recombinant lentivirus containing the recombinant expression vector according to claim 2.

5. A recombinant cell containing the myeloid-specific promoter according to claim 1.

6. A method for preparing the recombinant cell according to claim 5, comprising:
introducing a recombinant expression vector comprising a myeloid-specific promoter comprising a nucleic acid sequence as shown in SEQ ID NO: 1 or SEQ ID NO: 2 into a host cell to obtain the recombinant cell.

7. The method according to claim 6, comprising the following steps:
(1) constructing a lentiviral vector comprising the myeloid-specific promoter;
(2) co-transfecting the lentiviral vector in step (1) and a packaging plasmid or packaging plasmids into a mammalian cell for lentiviral vector packaging; and
(3) introducing the packaged lentiviral vector in step (2) into a host cell to obtain the recombinant cell.

8. The method according to claim 7, wherein step (1) of constructing the lentiviral vector comprises: inserting the myeloid-specific promoter and a CYBB gene into a pTYF lentiviral vector.

9. A pharmaceutical composition, comprising the myeloid-specific promoter according to claim 1;
wherein the pharmaceutical composition further comprises any one or a combination of at least two of a pharmaceutically acceptable carrier, excipient or diluent.

10. (canceled)

11. The recombinant expression vector according to claim 2, wherein the recombinant expression vector comprises a viral vector or a plasmid vector comprising the myeloid-specific promoter.

12. The recombinant expression vector according to claim 11, wherein the viral vector comprises a pTYF lentiviral vector.

13. The recombinant expression vector according to claim 3, wherein the CYBB gene comprises a nucleic acid sequence as shown in SEQ ID NO: 3.

14. The method according to claim 6, wherein the introduction is carried out by a method which comprises any one of electrical gene transfer, a viral vector system, a non-viral vector system or gene gun injection.

15. The method according to claim 6, wherein the host cell comprises a hematopoietic stem cell.

16. The method according to claim 7, wherein the packaging plasmids in step (2) comprise pNHP and pHEF-VSVG; and the mammalian cell in step (2) comprises a 293T cell.

17. A method for treating chronic granulomatous disease, comprising: administering the myeloid-specific promoter according to claim 1 to a patient in need thereof.


SEQUENCE LISTING (5 sequences)

SEQ ID NO: 1 - DNA, 778 bp, Artificial sequence
  myeloid-specific promoter
acttgtacagcttcacagggctccatgcttagaaggaccccacacttagtttaatgttctgctgtcatcatcttgatattcttaatttttaaataaagggcctatcgttttcattttttactgggccttgcaaattatgtagctggttctgtatgccaggagagaagttggaagtaaaatggtattccaggaccaggaggcattctggcagagtgaaagaacatgtgatttggagtccatggggatgggtttaaatttcagctttccactaatttgctttgtgatactgagtatttccttttatccctcagaggctctgtttctcaattttgactacgggttttttcattagataatgtctcagttctggtattccaggtttccctcaattattctgggaaaacctccttgacccacaggcagagcctagggcagccaggtgctttctactctctctctctctgcagcttggaaagttagtgtctgttgaaggtcagctgggagttggtggaggcagggcagtggcctgctactattgctgcagtagcagaccctttcacaacagcattgttttgtcattttgcatccagatttccgttggctaacctcagtcttatcttcctcatttctgtttcctgttgaagacaccaagggcccttcaaaacacagaagcttcttgctcacggcagaaagcccaattccatctggcccctgcaggttggctcagcactggggaatcagagtcccctccatgaccaaggcaccactccactgacag

SEQ ID NO: 2 - DNA, 861 bp, Artificial sequence
  myeloid-specific promoter
tagccatttgctggaccaaatctggagggagaacccctaaaacccctaagtgaggttgcccagggggttgtccccaggtggggggaagcaggggagagaaaatggtagccatttttacattgttttgtatagtatttattgattcaggaaacaaacacaaaattctgattataatgacttggaaactgcctgtttgggcttctcatttcttacctccccttccctctcccacctgctactgggtgcatctctgctccccccttccccagcagatggttacctttgggctgttgctttcttgtcaccatctgagttctcagacgctggaaagccatgttctcggctctgtgaatgacaatgctgactggagtgctgcccctctgtaaagggctgggtgtggatggtcacaagcccctcacatgcctcagccaagaggaagtagtacaggggtcagcccagaggtccaggggaaaggagtggaaaccgatttccccaccaagggaggggcctgtacctcagctgttcccatagctacttgccacaactgccaagcaagtttcgctgagtttgacacatggaaccctgtggatcaactgccctaggactccgtttgcacccatgtgacactgttgactttgccctgatgaagcagggccaacagtcccctaacttaattacaaaaactaatgactaagagagaggtggctagagctgaggcccctgagtcaggctgtgggtgggatcatctccagtacaggaagtgagactttcatttcctcctttccaagagagggctgagggagcagggttgagcaactggtgcagacagcctagctggactttgggtgaggcggttcagcc

SEQ ID NO: 3 - DNA, 1713 bp, Artificial sequence
  CYBB
atggggaactgggctgtgaatgaggggctctccatttttgtcattctggtttggctggggttgaacgtcttcctctttgtctggtattaccgggtttatgatattccacctaagttcttttacacaagaaaacttcttgggtcagcactggcactggccagggcccctgcagcctgcctgaatttcaactgcatgctgattctcttgccagtctgtcgaaatctgctgtccttcctcaggggttccagtgcgtgctgctcaacaagagttcgaagacaactggacaggaatctcacctttcataaaatggtggcatggatgattgcacttcactctgcgattcacaccattgcacatctatttaatgtggaatggtgtgtgaatgcccgagtcaataattctgatccttattcagtagcactctctgaacttggagacaggcaaaatgaaagttatctcaattttgctcgaaagagaataaagaaccctgaaggaggcctgtacctggctgtgaccctgttggcaggcatcactggagttgtcatcacgctgtgcctcatattaattatcacttcctccaccaaaaccatccggaggtcttactttgaagtcttttggtacacacatcatctctttgtgatcttcttcattggccttgccatccatggagctgaacgaattgtacgtgggcagaccgcagagagtttggctgtgcataatataacagtttgtgaacaaaaaatctcagaatggggaaaaataaaggaatgcccaatccctcagtttgctggaaaccctcctatgacttggaaatggatagtgggtcccatgtttctgtatctctgtgagaggttggtgcggttttggcgatctcaacagaaggtggtcatcaccaaggtggtcactcaccctttcaaaaccatcgagctacagatgaagaagaaggggttcaaaatggaagtgggacaatacatttttgtcaagtgcccaaaggtgtccaagctggagtggcacccttttacactgacatccgcccctgaggaagacttctttagtatccatatccgcatcgttggggactggacagaggggctgttcaatgcttgtggctgtgataagcaggagtttcaagatgcgtggaaactacctaagatagcggttgatgggccctttggcactgccagtgaagatgtgttcagctatgaggtggtgatgttagtgggagcagggattggggtcacacccttcgcatccattctcaagtcagtctggtacaaatattgcaataacgccaccaatctgaagctcaaaaagatctacttctactggctgtgccgggacacacatgcctttgagtggtttgcagatctgctgcaactgctggagagccagatgcaggaaaggaacaatgccggcttcctcagctacaacatctacctcactggctgggatgagtctcaggccaatcactttgctgtgcaccatgatgaggagaaagatgtgatcacaggcctgaaacaaaagactttgtatggacggcccaactgggataatgaattcaagacaattgcaagtcaacaccctaataccagaataggagttttcctctgtggacctgaagccttggctgaaaccctgagtaaacaaagcatctccaactctgagtctggccctcggggagtgcatttcattttcaacaaggaaaacttctaa

SEQ ID NO: 4 - DNA, 60 bp, Artificial sequence
  splice donor site
ggcaagaggcgaggggcggcgactggtgagtacgccaaaaattttgactagcggaggcta

SEQ ID NO: 5 - DNA, 60 bp, Artificial sequence
  splice donor site
ggcaagaggcgaggggcggcgactgcagagtacgccaaaaattttgactagcggaggcta